(19) United States
(12) Patent Application Publication
(10) Pub. No.: US 2006/0229321 A1
(43) Pub. Date: Oct. 12, 2006

(54) Rosuvastatin in pre demented states

(76) Inventors: Hans Basun, Sodertalje (SE); Timothy Piser, Wilmington, DE (US); Ihor Rak, Wilmington, DE (US)

Correspondence Address: ASTRA ZENECA PHARMACEUTICALS LP;GLOBAL INTELLECTUAL PROPERTY, 1800 CONCORD PIKE, WILMINGTON, DE, 19850-5437, US

(21) Appl. No.: 10/492,971

(22) PCT Filed: Oct. 18, 2002
(86) PCT No.: PCT/SE02/01911

§ 371 Date: Feb. 13, 2006

(30) Foreign Application Priority Data
Oct. 19, 2001 (SE) 0103509-6

(51) Int. Cl. A61K 31/522

(52) U.S. Cl. 514/263.38

(57) ABSTRACT

Provided is a method of preventing dementia in a patent comprising administering to a patient at risk of developing dementia an effective amount of rosuvastiatin or its pharmaceutically acceptable salt.

### BACKGROUND OF THE INVENTION

Rosuvastatin (defined herein to include its pharmaceutically acceptable salts such as for example the sodium or calcium salt, as described in U.S. Pat. No. 5,260,440 in examples 1 and 7 respectively). The calcium salt of rosuvastatin is represented by the chemical name bis[(E)-7-[4-(4-fluorophenyl-6-isopropyl-2-[methyl(methylsulfonyl)amino]pyrimidin-5-yl]-(3R,5S)3,5-dihydroxyhept-6-enoic acid] calcium salt and is the preferred compound for the invention described herein. U.S. Pat. No. 5,260,440 is incorporated herein by reference. Rosuvastatin is a statin which inhibits 3-hydroxy-3-methyl-glutaryl coenzyme A (HMG-CoA) reductase. Rosuvastatin is useful in the treatment of ailments such as hypercholesterolemia, hyperlipoproteinemia, and atherosclerosis

A recent study concludes that the use of statins could substantially reduce the risk of dementia in the elderly. Zornberg et al., D A. Statins and the Risk of Dementia Lancet 356:1627-1631 (Nov. 11, 2000). The authors admit that what they have identified is an association and not a casual link. Treatment of hypercholesterolemia with Lovastatin was observed to result in small performance decrements on neuropsychological tests of attention and psychomotor speed. Am J. Med. 2000:108:538-547 (2000). Other studies have found no effect on cognitive function following treatment with statins Id. At 542. The use of Rosuvastatin for the prevention of dementia has not previously been described.

### SUMMARY OF THE INVENTION

Provided herein is a method of preventing dementia in a patient comprising administering to a patient at risk of developing dementia an effective amount of rosuvastatin and the use of rosuvastatin or its pharmaceutically acceptable salt for the manufacture of a medicamnent for administration to a patient at risk of developing dementia.

### DETAILED DESCRIPTION OF THE INVENTION

Dementia, for purposes of the present invention includes Alzheimer's disease (AD), vascular dementia and mixed cases. The early stages of dementia has to some degree been elucidated and defined. For example, studies have established a group of individuals that are at risk of developing dementia. These individuals suffer from mild cognitive impairment (MCI). MCI refers to a clinical state wherein the individuals are memory impaired but do not meet the clinical criteria for dementia. Petersen, et al., Practice parameter: Early detection of dementia: Mild cognitive impairment (an evidence-based review), Neurology, 56:1133-1142 (2001). The criteria used to establish MCI is as follows: 1) the presence of a subjective memory complaint, preferably corroborated by an informant; 2) preserved general intellectual functioning as estimated by performance on a vocabulary test; 3) demonstration of a memory impairment by cognitive testing; 4) intact activities of daily living; and 5) absence of dementia.

Another group of individuals that are at risk for developing dementia are those in a pre-demented state found with age associated cognitive decline (AACD) which is generally defined by a decline of more than one standard deviation in any are of cognitive functioning in comparison with age matched controls. K. Ritchie. et al., Classification criteria for mild cognitive impairment: A population-based validation study, Neurology 56:3742 (2001). Ritchie et al., argues that AACD has a higher predictive validity for dementia onset. Id. at 40.

A further pre-demented condition may be determined by examining the following criteria: 1) subjective cognitive complaint: involves-substantial cognitive impairment reported by patient and proxy and it may include one or more cognitive domains, but not necessarily memory; 2) objective cognitive impairment: established by a battery of neuropsychological tests, preferably those that can be followed for at least 2 years and the tests should cover memory, attention, visuospatial abilities, and executive function; 3) global cognition scale: a Global Deterioration Scale (GDS)'suggested with a score of 3; and 4) not demented according to DSM-III-R criteria.

Yet another pre-demented state is describe in Graham et al., Prevalence and severity of cognitive impairment with and without dementia in an elderly population, Lancet 349:1793-6 (1997).

A pre-demented state may also be evaluated using a measurement of vascular cognitive impairment which is described by Wentzel et al, Progression of impairment in patients with vascular cognitive impairment without dementia, Neurology 2001; 57:7146 (2001). In this study, it was found that the 46% of the participants found to have vascular CIND developed dementia.

In carrying out the present invention, a clinician would for example use one of the above methods to determine if a patient is at risk for developing dementia. In another aspect of the present invention, a patient found to fit the criteria for a pre-demented condition, e.g., as defined above, would be a particular example of a patient suitable for administration of an effective amount of rosuvastatin. An effective amount of rosuvastatin is an amount sufficient to symptomatically relieve cognitive symptoms in a patient. This may be shown for example by a slowing of the progression or worsening of cognitive symptoms or by reducing the risk of patients with cognitive symptoms form getting worse (progressing to dementia).

Practitioners may use known methods to optimise the use of rosuvastatin to prevent dementia. For example, skilled practitioners may use clinical studies as a method to maximise the efficacy of the drug. Accordingly, the dose and therapeutic effect of rosuvastatin may be demonstrated by conventional controlled clinical trials in subjects with a pre-demented condition. The therapeutic effect of rosuvastatin in these patients will be shown via symptomatic relief of cognitive symptoms, slowing of progression of worsening cognitive symptoms, or reducing the risk of patients with cognitive symptoms form getting worse (progressing to dementia or worsening degree of dementia).

Rosuvastatin can be administered orally or parentally using known methods. If orally administered, rosuvastatin may be provided in the form of a tablet, powder, capsules, granules, aqueous or oily suspensions or liquid form such as syrup or elixir. If parenterally administered, it may typically be provided in the form of an aqueous or oily suspension. Conventional methods may be used to formulate rosuvastatin or its pharmaceutically acceptable sale for example, excipients, binders, lubricants, aqueous or oily solubilizers, emulsifiers, and suspending agents. Preservatives and stabilizers can be further used. Preferred formulation may be found for example in PCT application No.: WO 01/54668, incorporated herein by reference. The dosage would vary with the administration route, age, weight, condition, and the kind of disease of the patients, but would typically be 0.5-200 mg/day. If an oral dosage form is used a dosage of 1-100 mg/day, preferably 1-80 mg/day would be used. If given parentally, the dosage may be 0.5-50 mg/day. The dosage may be given in single or divided doses. A typical dosing regimen for rosuvastatin would be oral once a day from 1 to 80 mg in patients.

Studies in the mouse have demonstrated that subcutaneous administration of 2 or 20 mg/kg of rosuvastatin (calcium salt) for 14 days increased the expression and activity of eNOS and reduced the volume of infarct resulting from a subsequent cerebral ischemia caused by middle-cerebral artery occlusion (MCAO). The studies were generally carried out according to the methods set forth in M. Endres et al., Stroke protection by 3-hydroxy-3-methylglutaryl (HMG)-CoA reductase inhibitors mediated by endothelial nitric oxide synthase, Proc. Natl. Acad. Sci. USA, 95:8880-8885 (1998). In many cases dementia is known to result from the cumulative neurodegenerative effects of strokes. These can be major strokes or sub-clinical strokes, and result in a heterogeneous group of dementias commonly called vascular dementia (VAD). In this study rosuvastatin protected the brain in mice from cerebral ischemia A mechanism by which rosuvastatin may prevent dementia is by protecting the brain from cerebral ischemia.

While not wishing to be bound by any theory it is believed that there are several mechanisms of action whereby rosuvastatin might prevent dementia. Endothelial nitric oxide synthase (eNOS) is expressed by endothelial cells of the arterial vasculature. eNOS liberates nitric oxide (NO) by converting the amino acid arginine to citrulline. NO causes relaxation of vascular smooth muscle closely apposed to the endothelial cells, and is thus a potent vasodilating agent. Dilation of cerebral vasculature leads to increased cerebral blood flow and protects the brain from ischemic insults.

Mutations in the genes for the amyloid precursor protein (APP) and presenilin-1 (PS-1) cause increased brain levels of the peptide amnyloid-β (Aβ), and are the cause of familial Alzheimer's Dementia (fAD). The brains of Alzheimer's patients lacking mutations in these genes exhibit fibrillar plaques largely composed of Aβ, just as do the brains of fAD patients. Thus, increased levels of Ab in the brain is thought to cause both the deposition of Aβ into plaques (amyloidosis) and Alzheimer's Dementia (AD). The majority of demented patients exhibit evidence of both amyloidosis and cerebral ischemia. In fact, patients diagnosed with probable AD who died with both amyloid plaques and evidence of minor vascular ischemia (small “lacunar” infarcts), had much worse cognitive function than other patients with the same number of amyloid plaques. Thus, by protecting against cerebral ischemia by the mechanism described above, rosuvastatin may prevent both VAD, AD, and mixed AD/VAD.

Another mechanism by which rosuvastatin might prevent dementia is by reducing brain Aβ levels. One mechanism whereby rosuvastatin might reduce brain Aβ levels is by increasing the removal of Aβ from the brain. The cell-surface receptor LRP-1 (LDL receptor related protein-1) has been shown to mediate the transport of Aβ bound to the LRP-1 ligands apolipoprotein E (ApoE) and β-2 macroglobulin (□2M). Polymorphisms associated with decreased expression of LRP-1 are have been associated with increased risk of AD. Allelic inheritance of the ApoE4 allele of the LRP-1 ligand ApoE has also been linked to an increased risk of AD. Further evidence suggests that LRP-1 is expressed in endothelial cells of the cerebral vasculature, and that Aβ is normally extruded from the brain by transport across the endothelial cell layer dependent on the function of LRP-1. Thus LRP-1/ApoE may represent an important route for the removal of AP from the brain. The LRP-1 gene, like the closely related LDLR gene, contains a DNA sequence called the sterol responsive element (SRE1). This gene sequence causes the transcription of a gene to be responsive to cellular levels of sterols related to cholesterol. When cell sterol levels decline, the transcription of genes containing an SRE is increased. In fact, liver LRP-1 mRNA levels have been shown to increased following administration of a cholesterol-lowering dose of a statin. Rosuvastatin decreases the biosynthesis of cholesterol. By reducing the biosynthesis of cholesterol, rosuvastatin may decrease endothelial cell sterol levels, thereby increasing the transcription of the LRP-1 gene. The resulting increased expression of the LRP-1 cell-surface receptor may increase the ligand-mediated extrusion of Aβ from the brain. Statins are further known to increase expression of ApoE. Increased expression of ApoE could further increase ApoE/LRP-1 mediated extrusion of Aβ from the brain. Thus another mechanism by which rosuvastatin may prevent dementia is by increasing LRP-1/ApoE dependent extrusion of Aβ from the brain.

Rosuvastatin has been shown to be superior to other coenzyme A (HMG-CoA) reductase inhibitors in reducing cholesterol in patients which is unexpected particularly in its ability to prevent dementia. Thus, it is surprising and unexpected that rosuvastatin provides a method for preventing dementia in a patient at risk of developing dementia such as patients shown to have an observed pre-demented state.

We claim:

1. A method of preventing dementia in a patient comprising administering to a patient at risk of developing dementia an effective amount of rosuvastatin or its pharmaceutically acceptable salt.

2. Use of rosuvastatin or its pharmaceutically acceptable salt for the manufacture of a medicament for administration to a patient at risk of developing dementia.
